(19) United States
(12) Patent Application Publication
(10) Pub. No.: US 2026/0157779 A1
(43) Pub. Date: Jun. 11, 2026

(54) FEMORAL NECK TROCHANTERIC NAIL

(71) Applicant: GLOBUS MEDICAL, INC., AUDUBON, PA (US)

(72) Inventors: Daniel Myers, Conshohocken, PA (US); Jonan Philip, Phoenixville, PA (US); John Peterson, Conshohocken, PA (US)

(21) Appl. No.: 18/976,688

(22) Filed: Dec. 11, 2024

(51) Int. Cl. A61B 17/72

(52) U.S. Cl. CPC A61B 17/7241; A61B 17/725

(57) ABSTRACT

A femoral neck trochanteric nail makes use of a dual, lag-screw/anti-rotation screw assembly to provide two, spaced fixation points locatable across a femoral neck in conjunction with a corresponding hip procedure. The anti-rotation screw may be advanced through a bore in the lag screw after the lag screw has been suitably fixed to a trochanteric nail received at an intramedullary location. The lag screw and anti-rotation screw construct may be oriented in different positions to have corresponding fixation points at different angular locations after fixation.

### FIELD

This disclosure relates generally to the orthopedic surgical field and to trochanteric nailing systems, in particular, such as a femoral neck trochanteric nail.

### BACKGROUND

Trochanteric nails and their related components are indicated for treatment of fractures in adults and adolescents for various fractures, including basal neck fractures, fixation of stable and unstable intertrochanteric, pertrochanteric, and subtrochanteric fractures, pathologic fractures (including prophylactic use) in both trochanteric and diaphyseal regions, combinations of pertrochanteric, intertrochanteric, basal neck fractures, long subtrochanteric fractures, tumor resections, fractures resulting from trauma, nonunions, malunions, and revision procedures.

Treatment of these or other femoral fractures, such as transcervical and subcapital femoral neck fractures, may benefit from the use of trochanteric nails with features which offer the surgeon or medical practitioner different options, depending on fracture pattern, patient anatomy, or any number of other considerations.

### SUMMARY

In one suitable implementation, a femoral neck trochanteric nail is disclosed and may be used on a patient in association with a hip surgery. The femoral neck trochanteric nail includes not only a trochanteric nail portion, but also an anti-rotation screw and a lag screw adapted to receive the anti-rotation screw therethrough. To that end, the lag screw has an anti-rotation screw bore through which the anti-rotation screw may be selectively inserted. The anti-rotation screw bore extends at an angle to the longitudinal axis of the lag screw and can be advanced through the bore and locked relative to the bore through a pair of corresponding locking threads. The anti-rotation screw increases stability against rotational forces about the femoral neck when the femoral neck trochanteric nail is used in an associated procedure.

The lag screw is insertable into a corresponding lag screw bore defined in the trochanteric nail portion. The lag screw may be suitably advanced during the procedure and have its position fixed by means of a set screw which engages the lag screw from the trochanteric nail portion. Once the lag screw has been suitably fixed, the anti-rotation screw may be advanced through the anti-rotation screw bore as described above and into portions of the femoral head to create two, spaced fixation points and increase stability against rotation forces.

In certain implementations, the femoral neck trochanteric nail may include a trochanteric nail in the form of a cephalomedullary nail.

In still other implementations, the lag screw and the anti-rotation screw each have corresponding heads which have drive-receiving portions therein. A suitable driver or similar implement may be associated with the drive receiving portions to selectively advance the lag screw or the anti-rotation screw during associated surgical procedures.

In still further implementations, it may be desirable to have only a single lag screw bore formed in the trochanteric nail portion. In this implementation, only a single one of the lag screws is needed for the associated procedure, yet by virtue of the anti-rotation screw being received through the corresponding lag screw bore, the lag screw and the anti-rotation screw provide two fixation points in the femur to increase stability against rotational forces through a single bore in the trochanteric nail portion.

Still further implementations may include an engagement portion in the lag screw, that is, a portion along the length of the lag screw which is received in the lag screw bore of the trochanteric nail portion and selectively positionable relative to the lag screw bore for engagement by a set screw. The engagement portion of the lag screw may be formed to include a plurality of set screw grooves defined at corresponding angular locations about the diameter of the engagement portion. By virtue of the anti-rotation screw being received through the anti-rotation screw bore of the lag screw at a corresponding angle, and the ability of the lag screw itself to be fixed at an orientation corresponding to one of the set screw grooves, angular orientation of the anti-rotation screw may be varied relative to the orientation of the lag screw by suitable selection of one of the set screw grooves and, thereby, the relative locations of the corresponding, two fixation points are correspondingly varied.

In one possible implementation of the foregoing, there are four of the set screw grooves placed at 90° intervals about the diameter of the engagement portion. As such, an engagement of the lag screw at one of the four set screw grooves corresponds to four different trajectories of the anti-rotation screw. Such trajectories may allow for the anti-rotation screw to be oriented in a superior, posterior, or anterior position relative to the lag screw and the patient anatomy.

In another implementation, the anti-rotation screw and the lag screw comprise a femoral neck screw assembly which can be used in association with a selected trochanteric nail.

### BRIEF DESCRIPTION OF THE DRAWINGS

The foregoing implementations and their features will be better understood with reference to the description herein and the drawings, in which:

FIG. 1 is a side, elevational view of a femoral neck trochanteric nail according to one possible implementation of the present disclosure;

FIG. 2 is an enlarged, cross-sectional view of another potential implementation of a femoral neck trochanteric nail having the femoral neck trochanteric nail assembly at a different orientation than that shown in the implementation of FIG. 1;

FIG. 3 is an isometric view of a lag screw of the femoral neck trochanteric nail of FIGS. 1 and 2;

FIG. 4 is a cross-sectional view of the lag screw of FIG. 3;

FIG. 5 is a side elevational view of an anti-rotation screw suitable for use in implementations of a femoral neck trochanteric nail according to the present disclosure;

FIG. 6 is a side elevational view of another possible implementation of a femoral neck trochanteric nail according to the present disclosure, with the lag screw and the anti-rotation screw at another orientation from those previously illustrated in FIGS. 1 and 2.

### DETAILED DESCRIPTION

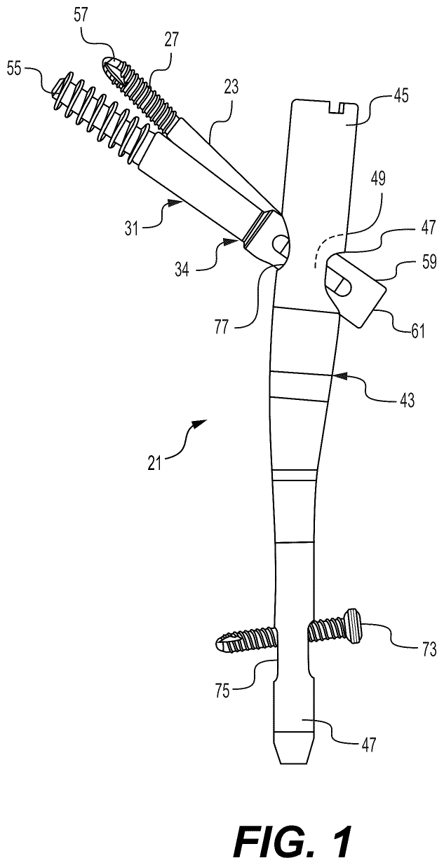

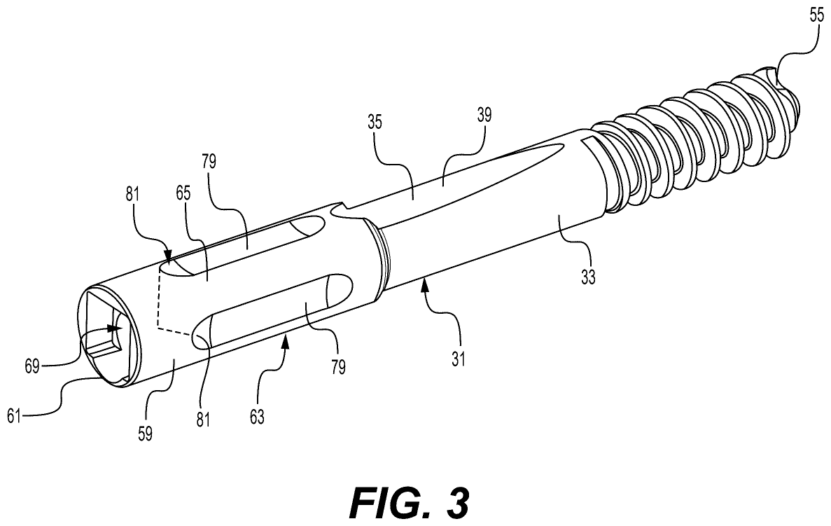

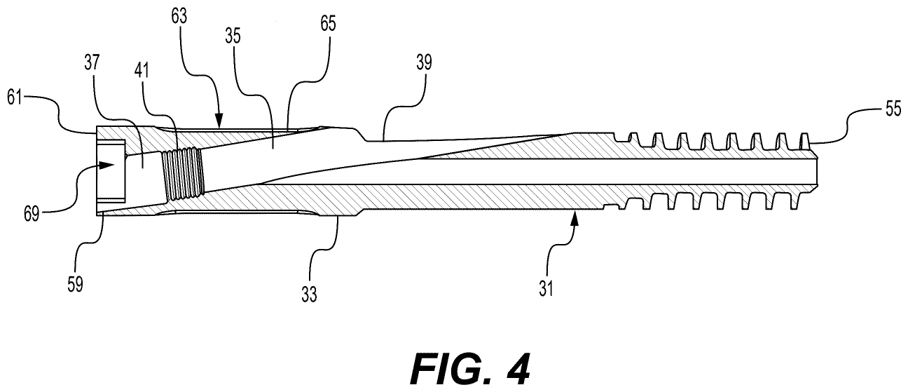

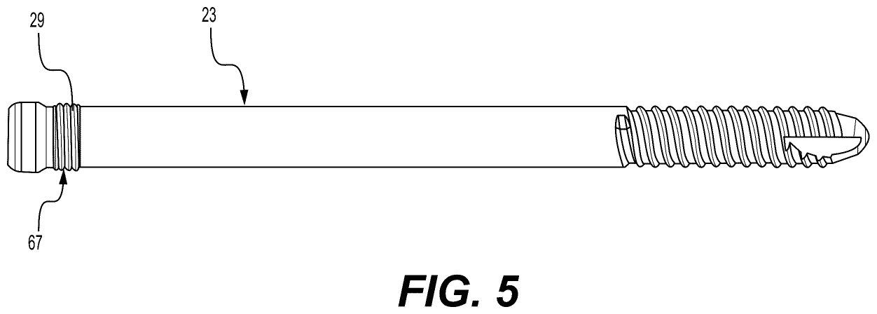

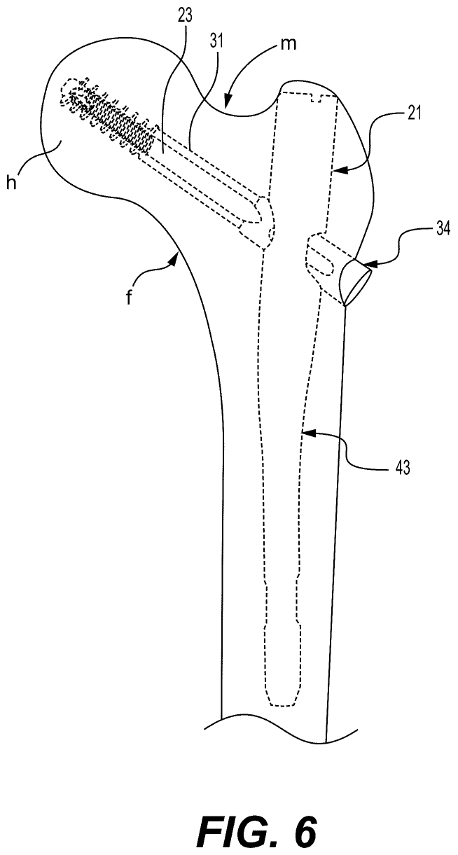

Referring to FIGS. 1-6 a femoral neck trochanteric nail 21 is useful on a patient in association with a surgical procedure of the hip. In the illustrated implementations, the femoral neck trochanteric nail 21 makes use of three main components, a trochanteric nail portion 43 which is sized and otherwise configured for intramedullary insertion in any number of associated hip surgeries. Trochanteric nail portion 43 is operatively associated with two other components of note, namely, a lag screw 31 which is received in a lag screw bore 49 defined in the trochanteric nail portion 43, and an anti-rotation screw 23 which is received in an anti-rotation screw bore 35 formed in lag screw 31. Trochanteric nail portion 43 likewise includes a set screw 51 threadably received in a set screw opening 53 defined in trochanteric nail portion 43 at a proximal end 45 thereof, as well as a distal locking screw 73 selectively receivable in a distal slot 75 at the distal end 47 of trochanteric nail portion 43.

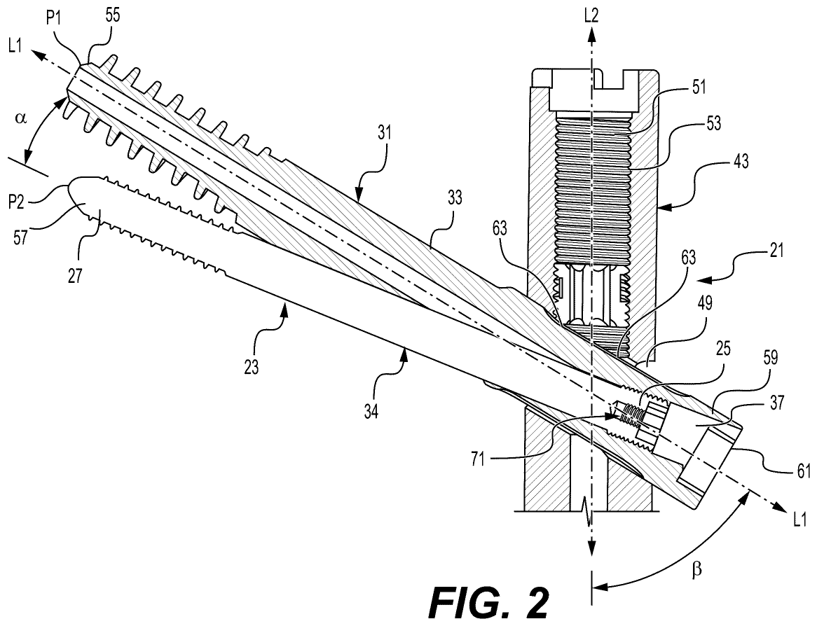

Lag screw 31 and anti-rotation screw 23 are sized so that, when fixed relative to trochanteric nail portion 43, lag screw 31 and anti-rotation screw 23 each may be suitably advanced to extend across femoral neck n of the patient. As shown in FIGS. 1 and 2, distal ends 55, 57 of lag screw 31 and anti-rotation screw 23, respectively, define two, spaced, fixation points P1, P2, operatively engaging femoral head h. The two fixation points P1, P2 act to increase stability of femoral neck n against potential rotational forces, such as those which may be experienced post operatively by the patient.

Lag screw 31 has a longitudinal axis L1 and an outer surface 33. Outer surface 33 in the illustrated implementation is substantially cylindrical and terminates in a series of engagement threads at distal end 55 of lag screw 31. Anti-rotation screw bore 35 extends at an angle α to longitudinal axis L1 and extends between respective first and second openings 37, 39 defined in the foregoing outer surface 33 lag screw 31.

Anti-rotation screw bore 35 is sized to slidably receive anti-rotation screw 23 therethrough. Anti-rotation screw bore 35 also includes locking threads 41 defined on the inner surface of bore 35, and which are adapted to threadably engage corresponding locking heads 29 formed at the proximal end of anti-rotation screw 23. As such, upon sufficient advancement, anti-rotation screw 23 may be fixed in position relative to lag screw 31 by means of mating locking threads 29, 41.

Lag screw 31 has a proximal end 49 and a lag screw head 61 disposed at proximal end 59. First opening 37 of anti-rotation screw bore 35 is defined at lag screw head 61, whereas second opening 39 of anti-rotation screw bore 35 is defined distally, that is, at a distal location relative to lag screw head 61. Second opening 39 is located on outer surface 33 corresponding to the angle α of lag screw bore 35.

Lag screw 31 has an engagement portion 63 located between first and second openings 37, 39 and engageable by set screw 51 when set screw 51 is threadably advanced through set screw opening 53 defined in trochanteric nail portion 43. In the illustrated embodiment, engagement portion 63 has a suitable diameter and an outer engagement surface 65 positionable at set screw opening 53. Engagement surface 65 may have a plurality of set screw grooves 79 defined therein at corresponding angular locations 81 about the diameter of engagement surface 65 and engagement portion 63. Each of the angular locations 85 or set screw grooves 79 correspond to an orientation of anti-rotation screw bore 35 relative to longitudinal axis L1 of lag screw 31. Once anti-rotation screw 23 has been received in lag screw 31 through bore 35, anti-rotation screw 23 will have the same corresponding angular orientation.

Lag screw 31 is rotatable relative to set screw 51 and lag screw bore 49 of trochanteric nail portion 43 to align a selected one of set screw grooves 79 with set screw 51. As such, in one suitable workflow or procedure, once the selected set screw groove 79 has been aligned, it may be fixed by suitable advancement of set screw 51 and thereby establish the corresponding angular orientation of anti-rotation screw bore 35, as well as that of anti-rotations screw 23 after it has been advanced through bore 35. By fixing the angular orientation of anti-rotation screw 23 relative to lag screw 31, the corresponding locations of fixation points P1, P2 are likewise determined. Accordingly, for certain procedures, if desired, a surgeon or other medical practitioner may select certain locations for fixation points P1, P2 within femur f, particularly femoral head h, to achieve desired objectives for the corresponding hip surgery.

In the illustrated implementation, there are four of the set screw grooves 79 spaced at 90° intervals about the diameter of engagement portion 63, such locations corresponding to four different potential trajectories for anti-rotation screw 23. By way of example only and not limitation, the four potential trajectories may correspond to superior, posterior, and anterior location of the fixation point of anti-rotation screw 23 relative to lag screw 31 and likewise relative to femur f. Other angular orientations and numbers of grooves 79 are likewise suitable.

Proximal end 59 of anti-rotation screw 23 has an anti-rotation screw head 67 defined therein. Anti-rotation screw head 67 and lag screw head 61 are formed to include respective drive-receiving portions 69, 71. Such drive-receiving portions 69, 71 are configured to be engageable by suitable drivers or similar means to selectively advance lag screw 31 and anti-rotation screw 23, respectively, into engagement with suitable portions of femur f, such as its femoral neck n and femoral head h.

Femoral neck trochanteric nail 21, in the illustrated implementation, may comprise only a single lag screw bore 49 and a single, corresponding lag screw 31 to potentially simplify associated hip surgical procedures. For example, and not by way of limitation, the location and angular orientation of anti-rotation screw bore 35 through lag screw 31 permits the aforementioned, single lag screw bore 49 to be used to establish two corresponding fixation points P1, P2 at spaced locations, without necessitating use of another lag screw bore on the trochanteric nail portion 43.

Lag screw bore 49 may be formed to have flared openings 77 on the outer surface of trochanteric nail portion 43. Such flared openings 77 have diameters slightly larger than the diameter of engagement portion 63 of lag screw 31, which offer associated advantages in use of femoral neck trochanteric nail 21.

In one possible implementation, lag screw 31 and its associated anti-rotation screw 23 may be grouped into a femoral neck screw assembly 34 for use with known, trochanteric or cephalomedullary nails.

The available operations and use of femoral neck trochanteric nail 21 are apparent from the foregoing description. One suitable process for securing a trochanteric nail relative to the femoral neck of the femur of a patient may comprise a work flow as follows: trochanteric nail portion 43 is inserted at an intramedullary location of femur f. Thereafter, a lag screw is inserted into femur f and through the lag screw bore 49 of trochanteric nail portion 43. After insertion of lag screw 31, anti-rotation screw 23 is inserted into femur f and through anti-rotation screw bore 35 defined in lag screw 31. As described previously, the anti-rotation screw bore 35 is oriented at an angle relative to that of lag screw 31 so that the distal end of lag screw 31 and the distal end of anti-rotation screw 23, when received in the anti-rotation screw bore 35, define two spaced fixation points P1, P2 in operative proximity to femoral neck n. Among the surgical procedures which may benefit from such fixation points are treatments of transcervical and subcapital femoral neck fractures.

Further process steps may involve rotating the lag screw 31 after it has been suitably advanced to align a selected one of the grooves 79 with set screw 51. As such, once selection of one of grooves 79 has been accomplished, set screw 51 can be thereafter advanced to engage the selected one of the set screw groves 79 and lock lag screw 31 in place rotationally.

After locking lag screw 31 in the desired position by means of set screw 51, suitable advancement of ant-rotation screw 23 relative to lag screw 31 engages the respective threads 29, 41.

The foregoing steps of inserting or advancing lag screw 31 and anti-rotation 23 may be accomplished by suitable engagement of respective, drive portions 69, 71 located on respective heads 61, 67. At the proximal ends of lag screw 31 and anti-rotation screw 23, thereby driving into corresponding femur portions. The access to the drive portion of anti-rotation screw 23 has been facilitated by locating the anti-rotation screw opening 37 at the proximal end of lag screw 31 near its head 61.

Any number of variations to the foregoing workflows and still other workflows and procedures are contemplated.

In addition to the advantages apparent from the foregoing description, it will be appreciated that the femoral neck trochanteric nail of the present disclosure comprises a fixation system which is both low profile, streamlined, and dual in the sense of providing two fixation points.

As a further advantage, the weight-bearing functionality of the trochanteric nail has been combined with rotational stability of the dual screw construct of the trochanteric nail assembly 34.

In the illustrated implementation, four, distinct lag screw/locking screw positions have been made available to provide a variety of fixation options to surgeons and other medical professionals.

As a still further advantage, a single lag screw bore may be used in certain implementations which facilitates the use of either a standard single lag screw, or, alternately, the trochanteric nail assembly 34 comprising a lag screw, anti-rotation screw construct.

It will be further understood that various changes in the details, materials, and arrangements of the components disclosed herein may be made by those skilled in the art without departing from the spirit and scope of the present disclosure. Furthermore, one skilled in the art would appreciate that the illustrated and disclosed embodiments and variations thereof are non-limiting. It will also be appreciated that one or more features may be omitted from one or more other alternative embodiments without departing from the scope of the disclosure as expressed in the claims appended hereto.

What is claimed is:

1. A femoral neck trochanteric nail for use on a patient in an associated hip surgical procedure, comprising:
an anti-rotation screw to increase stability against rotational forces about the femoral neck, the anti-rotation screw having proximal and distal ends, the anti-rotation screw having first locking threads at the proximal end;
a lag screw adapted to receive the anti-rotation screw therethrough, the lag screw having a first, longitudinal axis and an outer surface, the lag screw having an anti-rotation screw bore extending at a first angle to the longitudinal axis between respective first and second openings defined on the outer surface, the anti-rotation screw bore sized to slidably receive the anti-rotation screw therethrough, the anti-rotation screw bore including second locking threads adapted to threadably engage the first locking threads of the anti-rotation screw and, upon sufficient advancement, to fix the position of the anti-rotation screw relative to the lag screw;
a trochanteric nail portion having proximal and distal nail ends and a longitudinal axis therebetween, the trochanteric nail portion extending over a length and sized for intramedullary insertion;
wherein the trochanteric nail portion has a lag screw bore defined therein and adapted to receive the lag screw therethrough, the lag screw bore extending at a second angle to the longitudinal axis of the trochanteric nail portion;
a set screw threadably receivable in a set screw opening defined in the trochanteric nail portion at the proximal nail opening, wherein the set screw opening is in communication with the lag screw bore, and wherein the set screw is adapted to be threadably advanced to engage the lag screw when the lag screw is received in the lag screw bore, thereby fixing the lag screw relative to the trochanteric nail portion; and
wherein the lag screw and the anti-locking screw are sized so that, when fixed relative to the trochanteric nail portion and the lag screw, respectively, the lag screw and the anti-locking screw extend across of the femoral neck of the patient and have respective distal ends at two, spaced fixation points operatively engaging the femoral head;
whereby the two fixation points increase the stability of the femoral neck against rotational forces.

2. The femoral neck trochanteric nail of claim 1, wherein the trochanteric nail portion comprises a cephalomedullary nail.

3. The femoral neck trochanteric nail of claim 1, wherein the proximal end of the lag screw has a proximal end and lag screw head disposed at the proximal end, wherein the first opening of the lag screw is defined at the head, wherein the second opening is defined at a distal location relative to the head, and wherein the lag screw has an engagement portion located between the first and second openings, the engagement portion having a diameter and an outer engagement surface positionable at the set screw opening and thereby engageable by advancement of the set screw.

4. The femoral neck trochanteric nail of claim 3, wherein the proximal end of the anti-rotation screw has an anti-rotation screw head, and wherein the lag screw head and the anti-rotation screw head include respective, drive-receiving portions.

5. The femoral neck trochanteric nail of claim 3, further comprising a distal locking screw, and wherein the trochanteric nail portion has a distal slot sized to receive the distal locking screw therethrough.

6. The femoral neck trochanteric nail of claim 3, comprising only a single one of the lag screws and only a single corresponding lag screw bore.

7. The femoral neck trochanteric nail of claim 3, wherein the openings of the lag screw bore comprise flared openings at respective locations on the outer surface of the trochanteric nail portion, the flared openings having diameters larger than the diameter of the engagement portion.

8. The femoral neck trochanteric nail of claim 3, wherein the engagement portion of the lag screw further comprises a plurality of set screw grooves defined at corresponding angular locations about the diameter of the engagement portion, each of the angular locations of the grooves corresponding to a corresponding orientation of the anti-rotation screw relative to the lag screw; wherein the lag screw is rotatable relative to the set screw to fix a selected one of the grooves with the set screw and thereby fix the corresponding angular orientation of the anti-rotation screw relative to the lag screw, thereby selecting the locations of the corresponding two fixation points.

9. The femoral neck trochanteric nail of claim 8, comprising four of the grooves spaced at 90-degree intervals about the diameter to correspond to four different trajectories for the anti-rotation screw, the trajectories including superior, posterior, and anterior.

10. A femoral neck screw assembly for use with a cephalomedullary nail on a patient in an associated hip surgical procedure, in which the cephalomedullary nail has proximal and distal nail ends and a longitudinal axis therebetween, and in which the cephalomedullary nail extends over a length, has a lag screw bore defined therein, and is sized for intramedullary insertion, the femoral neck screw assembly comprising:
an anti-rotation screw to increase stability against rotational forces about the femoral neck, the anti-rotation screw having proximal and distal ends, the anti-rotation screw having first locking threads at the proximal end;
a lag screw adapted to be received in the lag screw bore of the cephalomedullary nail;
wherein the lag screw is further adapted to receive the anti-rotation screw therethrough after the lag screw has been received in the cephalomedullary nail, the lag screw having a longitudinal axis and an outer surface, the lag screw having an anti-rotation screw bore extending at a first angle to the longitudinal axis between respective first and second openings defined on the outer surface, the anti-rotation screw bore sized to slidably receive the anti-rotation screw therethrough, the anti-rotation screw bore including second locking threads adapted to threadably engage the first locking threads of the anti-rotation screw and, upon sufficient advancement, to fix the position of the anti-rotation screw relative to the lag screw; and
wherein the lag screw and the anti-locking screw are sized so that, when fixed relative to the trochanteric nail portion and the lag screw, respectively, the lag screw and the anti-locking screw extend across of the femoral neck of the patient and have respective distal ends at two, spaced fixation points operatively engaging the femoral head;
whereby the two fixation points increase the stability of the femoral neck against rotational forces.

11. The femoral neck screw assembly of claim 10, for use with a cephalomedullary nail in which a set screw is threadably receivable in a set screw opening defined in the cephalomedullary nail and in communication with the lag screw bore, the femoral neck screw assembly further comprising a lag screw head defined at a proximal end of the lag screw, the first opening of the lag screw defined at the head, the second opening defined at a distal location relative to the head, and wherein the lag screw has an engagement portion located between the first and second openings, the engagement portion having a diameter and an outer engagement surface positionable at the set screw opening and thereby engageable by advancement of the set screw.

12. The femoral neck screw assembly of claim 11, wherein the proximal end of the anti-rotation screw has an anti-rotation screw head, and wherein the lag screw head and the anti-rotation screw head include drive-receiving portions.

13. The femoral neck screw assembly of claim 11, wherein the engagement portion of the lag screw further comprises a plurality of set screw grooves defined at corresponding angular locations about the diameter of the engagement portion, each of the angular locations of the grooves corresponding to a corresponding orientation of the anti-rotation screw relative to the lag screw; wherein the lag screw is rotatable relative to the set screw to fix a selected one of the grooves with the set screw and thereby fix the corresponding angular orientation of the anti-rotation screw relative to the lag screw, thereby selecting the locations of the corresponding two fixation points.

14. The femoral neck screw assembly of claim 13, comprising four of the grooves spaced at 90-degree intervals about the diameter to correspond to four different trajectories for the anti-rotation screw, the trajectories including superior, posterior, and anterior.

15. A process for securing a trochanteric nail relative to a femoral neck of a femur of a patient, the process comprising the steps of:
inserting the trochanteric nail at an intramedullary location of the femur, the trochanteric nail having a set screw received in a set screw bore and a lag screw bore defined through the trochanteric nail at a longitudinal location positionable in operative proximity to the femoral neck of the patient;
inserting a lag screw into the femur and through the lag screw bore; and
inserting an anti-rotation screw into the femur and through an anti-rotation screw bore defined in the lag screw, the bore oriented at an angle relative to the lag screw so that the distal end of the lag screw and the distal end of the anti-rotation screw when received in the anti-rotation screw bore define two, spaced fixation points in operative proximity to the femoral neck, the two fixation points increasing the stability of the femoral neck against rotational forces.

16. The process of claim 15, further comprising the steps of
rotating the inserted lag screw to align a selected one of a plurality of groove of the lag screw with the set screw; and
advancing the set screw to engage the selected one of the grooves to lock the lag screw in place rotationally;

17. The process of claim 16, further comprising including the step of locking the inserted anti-rotation screw relative to the lag screw.

18. The process of claim 17, wherein the step of locking the inserted anti-rotation screw comprises advancing the anti-rotation screw relative to the anti-rotation screw bore sufficiently to threadably engage anti-rotation screw threads disposed at the proximal end of the anti-rotation screw with corresponding threads disposed at the proximal end of the lag screw.

19. The process of claim 15, wherein the steps of inserting the lag screw and the anti-rotation screw comprise engaging respective drive portions at the proximal ends of the screws and the drive the screws into corresponding femur portions.

20. The process of claim 15, wherein the step of inserting the anti-rotation screw comprises inserting the anti-rotation screw through an opening of the anti-rotation screw bore located at a head of the lag screw.
